(19) United States
(12) Patent Application Publication
(10) Pub. No.: US 2015/0265208 A1
(43) Pub. Date: Sep. 24, 2015

(54) SYSTEMS AND METHODS FOR PAIN MEASUREMENT

(71) Applicant: ORIDION MEDICAL 1987 LTD., Jerusalem (IL)

(72) Inventor: Paul S. Addison, Midlothian (GB)

(73) Assignee: ORIDION MEDICAL 1987 LTD., Jerusalem (IL)

(21) Appl. No.: 14/218,499

(22) Filed: Mar. 18, 2014

(51) Int. Cl. A61B 5/00; A61B 5/145; A61B 5/053; A61B 5/021; A61B 5/024

(52) U.S. Cl. CPC A61B 5/4824; A61B 5/021; A61B 5/024; A61B 5/0531; A61B 5/14542

(57) ABSTRACT

Provided are systems and method for determination, measurement and management of pain of a subject based on measurements of various physiological parameters of the subject.

### TECHNICAL FIELD

The present disclosure generally relates to medical monitoring systems and methods of using the same. In particular, the disclosure is directed to medical monitoring systems and methods using the same for determination and measurement of pain.

### BACKGROUND

Medical monitoring devices are routinely used in various medical settings to obtain or measure physiological parameters relating to a patient's medical condition.

Pain is one of the most important, yet difficult, constructs to measure in clinical practice and research. By definition, pain is subjective and relies on the patient's self-report; measures of pain estimated by proxies, particularly health-care providers, invariably underestimate pain.

### SUMMARY

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other advantages or improvements.

According to some embodiments, there are provided methods and systems for measurement and/or management of pain of a subject in various health related conditions under various settings.

According to some embodiments, the methods and systems provided herein may be used for the measurement and/or assessment of pain of a subject and may optionally further provide pain management by controlling titration of sedation of the treated subject so as to reduce pain levels.

In some embodiments, the methods provided herein take use of measurements or related information obtained from one or more medical monitoring devices. In some embodiments, the medical monitoring devices may be selected from, but not limited to: capnograph, pulse oximeter, and measurements of skin humidity or conductivity. In some embodiments, the measurements or related information obtained from the medical monitoring devices are various physiological, parameters, such as, for example, but not limited to: breath related parameters (as determined, for example, based on CO2 measurements in exhaled breath), heart rate related parameters, blood related parameters, brain activity related parameters, skin related parameters (such as skin humidity, skin conductivity), body temperature, and the like, or combinations thereof.

According to some embodiments there is provided a system for measuring, assessing, determining and/or controlling pain, the system comprising one or more medical devices configured to measure one or more physiological parameters of a subject; and a processing unit capable of integrating the measured physiological parameters to determine the pain level of the subject based on the measured parameters. In some embodiments, the system comprises a combination of at least three medical monitoring devices. In some embodiments, the medical monitoring devices are selected from: a capnograph, a pulse oximeter and a skin humidity and/or conductivity measurement device. In some embodiments, the processing unit integrates more than one discrete physiological parameter. In some embodiments, the processing unit integrates at least two discrete physiological parameters. In some embodiments, the processing unit integrates at least three discrete physiological parameters. In some embodiments, at least one of the measured physiological parameters is expired CO₂.

In some embodiments, the measured parameters may be further manipulated and/or processed prior to or simultaneously while being integrated by the processing unit.

Surprisingly, the methods and systems disclosed herein, which take use of measurement of CO₂ in breath (expired CO₂) in combination with measurements of various additional physiological parameters and/or data derived therefrom (such as, skin humidity, heart rate, and the like), provide a more accurate and reliable means for determining, evaluating and/or controlling pain levels of the subject, which is indicated by changes in the measured parameters. Thus, the systems and methods disclosed herein advantageously provide the health care provider with an efficient, accurate, non-invasive and continuous means for measuring, evaluating and/or controlling the pain levels of a subject. The methods and systems disclosed herein advantageously further provide an accurate, non-subjective assessment and measurement of pain levels of a subject, in particular of subjects that are unable or at difficulty at providing their own indication as to their pain levels, such as, for example, sedated patients or intubated patients. The methods and systems disclosed herein advantageously may further allow titration of sedative substances administered to a subject on increasing pain so that lower doses of sedatives may be used. The methods and systems disclosed herein advantageously may further allow automatic titration of the sedatives, and/or may further allow issuing an indication or alert to the health care provider that titration of sedative substances is needed.

According to some embodiments, there is provided a system for determining pain level of a subject, the system may include: one or more medical monitoring devices configured to measure three or more physiological parameters of the subject, at least one of said parameters is expired CO2; a processing unit configured to integrate the one or more physiological parameters of the subject, to determine the pain level of the subject based on the measured parameters; and a signaling unit configured to issue an alert if the pain level of the subject increases.

According to some embodiments, the medical monitoring device may include capnograph, pulse oximeter, skin humidity measurement device, skin conductance measurement device, skin capacitance measurement device Electrocardiogram (ECG), Brain activity monitoring device, or combinations thereof.

According to some embodiments, the physiological parameters of the subject may include: skin humidity, skin conductance, skin capacitance, heart related parameters, blood related parameters, brain electrical activity, parameters derived therefrom, and combinations thereof.

According to some embodiments, the expired CO2 related parameters may include: CO2 concentration in breath, EtCO2, CO2 waveform, Respiration rate, data derived therefrom, and combinations thereof.

According to some embodiments, the heart related parameters may include heart rate, amplitude of cardiac pulses, Percent Modulation (PMod) of the cardiac pulses, or combinations thereof.

According to some embodiments, the blood related parameters may include blood pressure, SpO2 or both.

According to some embodiments, increase of the pain level is determined over a period of time and/or if the pain levels are determined to be higher than a predetermined threshold.

According to some embodiments, the alert issued by the signaling unit may include an audible alert, a visual alert, a tactile alert or combinations thereof.

According to some embodiments, the signaling unit may be further configured to control levels of sedative substances administered to the subject.

According to some embodiments, the system may further include a display unit configured to display one or more of: the measured parameters, the determined pain level, a change of one or more of measured parameters over time, a change of the determined pain levels over time, parameters derived from the measured parameters, parameters derived therefrom or combinations thereof.

According to some embodiments, there is provided a method for determining pain levels of a subject, the method comprising:
a) obtaining a measurement of three or more health related parameters of the patient, at least one of said parameters is expired CO2;
b) determining the pain level of the patient based on the measured parameters of the subject; and
c) issuing an alert if the pain levels increases over a time period and/or over a predetermined threshold.

Certain embodiments of the present disclosure may include some, all, or none of the above advantages. One or more other technical advantages may be readily apparent to those skilled in the art from the figures, descriptions, and claims included herein. Moreover, while specific advantages have been enumerated above, various embodiments may include all, some, or none of the enumerated advantages.

In addition to the exemplary aspects and embodiments described above, further aspects and embodiments will become apparent by reference to the figures and by study of the following detailed descriptions.

### BRIEF DESCRIPTION OF THE FIGURES

FIG. 1—a schematic presentation of a system for determining pain levels of a subject, according to some embodiments;

FIG. 2—a schematic a graph of amplitude of cardiac pulses in a photoplethysmograph signal over time, according to some embodiments;

FIG. 3—a schematic block diagram of steps in a method of determining pain levels of a subject, according to some embodiments.

### DETAILED DESCRIPTION

In the following description, various aspects of the disclosure will be described. For the purpose of explanation, specific configurations and details are set forth in order to provide a thorough understanding of the disclosure. However, it will also be apparent to one skilled in the art that the disclosure may be practiced without specific details being presented herein. Furthermore, well-known features may be omitted or simplified in order not to obscure the disclosure.

As referred to herein, the terms “user”, “medical user”, “health care provider” and “health care professional” may interchangeably be used. The terms may include any health care provider who may treat and/or attend to a patient. A user may include, for example, a nurse, respiratory therapist, physician, anesthesiologist, and the like. In some cases, a user may also include a patient.

As referred to herein, the terms “device”, “monitoring device” and “medical device” may interchangeably be used. Exemplary monitoring devices include such devices as, but not limited to: Capnograph, pulse oximeter, Electrocardiograph (ECG), skin humidity measuring device, thermometer, Brain electrical activity monitoring device (such as BIS), and the like.

As referred to herein, the term “physiological parameter” is directed to a health related parameter of the subject. The health related parameter may be directly and/or indirectly measured, detected and/or derived from a measurement of a medical monitoring device, for example, via an appropriate sensor. In some embodiments, the health related parameter may include such parameters as, but not limited to: breath related parameters (such as, for example, CO₂ related parameters, EtCO₂, O₂ related parameters, breath rate, breath cycle, respiration rate, and the like); heart related parameters (such as, pulse rate); blood related parameters (such as, blood pressure); temperature; skin related parameters (such as, skin humidity, skin conductance, skin capacitance, and the like); Brain related parameters (such as, brain activity); and the like; or combinations thereof.

As referred to herein, the terms “patient” and subject” may interchangeably be used and may relate to a subject being monitored by any monitoring device for any physical-condition related parameter and/or health related parameter. In some embodiments, the subject is sedated or anaesthetized. In some embodiments, the subject is ventilated. In some embodiments, the subject is unconscious.

As referred to herein, the term “waveform” is directed to a recurring graphic shape which may be realized by measuring a physiological parameter of a subject over time, such as, for example, concentration of CO₂ in breath, rate of breathing, electrocardiogram (ECG), plethysmograph, and the like. In some embodiments, a waveform is a medically, time resolved waveform. A waveform may have various characteristic parameters/features/factors that may be derived from the shape, dimension, rate or frequency, reoccurrences, and the like, and combinations thereof.

As referred to herein, the term “pattern(s)” relates to any identified/determined pattern over time, which is recurring known or known that may be produced when graphically displaying any of the waveforms or waveforms related factors/parameters. In some embodiments, a pattern may be predefined. In some embodiments, a pattern may be determined if it is clearly repeating itself for a given number of times over a given period of time.

As referred to herein, the terms ordinary, normal, typical, standard and common may interchangeably be used.

As referred to herein, the term “EtCO₂” relates to End tidal CO₂. The CO₂ is exhaled out of the body and the concentration of the exhaled CO₂, also known as end tidal CO₂ (EtCO₂) is an approximate estimation of the alveolar CO₂ pressure and thus of the arterial levels of CO₂. The values of EtCO2 may be measured in units of pressure, such as, for example, mmHg.

As referred to herein, the term “breath cycle” includes the stages of exhalation and inhalation. The breath cycle may be derived from a CO₂ waveform which depicts the change in expired CO₂ Volume over time, (EtCO₂). During a breath cycle, the levels of CO₂ initially increase as a result of CO₂ release from the airways, from what is known as the “dead space”, which is the space in which no gas exchange takes place. Then, the CO₂ rapidly reaches a plateau at high levels of CO₂, which corresponds to the release of CO₂ from the lungs, in the exhalation phase. A rapid decline in exhaled CO₂ proceeds the inhalation phase, characterized by absence/minute levels of CO₂.

As referred to herein, the term “Respiration Rate” (RR) may be defined as the number of breaths taken in a minute, and it may change under various physiological and medical conditions.

As referred to herein, the terms “Heart Rate” (HR) and “pulse rate” may interchangeably be used and may relate to the number of heart pulses (beats) in a minute. Pulse rate is usually considered to be a combination of left ventricular stroke volume, ejection velocity, the relative compliance and capacity of the arterial system, and the pressure waves that result from the antegrade flow of blood and reflections of the arterial pressure pulse returning from the peripheral circulation, and some or all of which may be effected by CO₂.

In some embodiments, the terms “calculated” and “computed” may interchangeably be used.

The terms “sedatives”, “sedative substances”, “sedative drugs”, “sedative agents”, “pain relieving substances” and “pain relieving drugs” may interchangeably be used. The terms are directed to substances or drugs that can reduce pain levels of a subject. In some embodiments, the sedatives may be administered by any administration route. In some embodiments, the administration of the sedative may be controlled by a pain management subunit. In some embodiments, the sedatives may be administered continuously or intermittently. In some embodiments, the amount (concentration) of the administered sedatives may be controlled based on pain levels of the subject. In some embodiments, the time period over which the sedatives are administered may be controlled based on pain levels of the subject.

Capnography is a non-invasive monitoring method used to continuously measure CO₂ concentration in exhaled breath. The CO₂, which is a constant metabolism product of the cells, is exhaled out of the body, and the concentration of the exhaled CO₂, also known as end tidal CO₂ (EtCO₂), is an approximate estimation of the arterial levels of CO₂. Capnograph (or capnometer) is a medical monitoring device that may be used for measuring the carbon dioxide (CO₂) content in inspired and expired air of a subject. It is a non-invasive device that measures the concentrations of respired gases.

Pulse oximeter is a type of oximeter, which is a medical monitoring device that may be used to determine oxygen saturation of the blood. Pulse oximeter may indirectly be used to measure the oxygen (O₂) saturation concentration and changes in blood volume in the skin (i.e., act as a photoplethysmograph). Pulse oximeter may also be used to measure the pulse rate of a subject. The term SpO₂ relates to the saturation of peripheral oxygen. It is a measurement of the amount of oxygen attached to the hemoglobin in red blood cells in the circulatory system. SpO₂ values are generally given as a percentage. SpO₂ may be monitored and measured by a Pulse Oximeter.

Skin humidity may be measured by various devices and means known in the art. The measurements provide an assessment as to the degree of humidity of the skin. The humidity may be may indirectly be determined according to changes in skin electrical conductivity (conductance) and/or electrical capacity (capacitance). In some embodiments, changes in skin humidity or conductance or capacitance are indicative of varying degrees of sweat (such as, for example, cold sweat) of the subject.

According to some embodiments, there are provided methods and systems for measurement and/or management of pain of a subject in various health related conditions under various settings.

According to some embodiments, the methods and systems provided herein may be used for the measurement and or assessment of pain of a subject and may optionally further provide pain management by controlling titration of sedation of the treated subject so as to reduce levels of pain.

In some embodiments, the systems and methods provided herein take use of measurements of physiological parameters (or information derived therefrom), obtained from one or more medical monitoring devices. In some embodiments, the medical monitoring devices may be selected from, but not limited to: capnograph, photoplethysmograph (such as, pulse oximeter), skin humidity or conductivity measuring device, thermometer, ECG, BIS, EEG, and the like. In some embodiments, the physiological parameters, or data derived therefrom, are such parameters as, for example, but not limited to: breath related parameters (as determined, for example, based on expired CO₂ (measurements of CO₂ in exhaled breath), heart related parameters (such as heart rate), blood related parameters (such as, blood pressure, oxygen saturation, blood gases), brain activity related parameters (such as brain electrical activity), skin related parameters (such as skin humidity or skin conductivity), body temperature, and the like, or combinations thereof.

According to some embodiments there is provided a system for measuring, assessing, determining and/or controlling pain levels of a subject, the system may include one or more medical monitoring devices configured to obtain, acquire or measure one or more physiological parameters of a subject; and a processing unit capable of integrating the measured physiological parameters to determine the pain level of the subject based on the measured parameters. In some embodiments, the system comprises a combination of at least two medical monitoring devices. In some embodiments, the system comprises a combination of at least three medical monitoring devices. In some embodiments, the medical monitoring devices may be selected from, but not limited to: a capnogrpah, a pulse oximeter, Brain activity monitor, a skin humidity and/or conductivity measurement device, or any combination thereof. In some embodiments, the processing unit may integrate more than one discrete physiological parameter. In some embodiments, the processing unit may integrate at least two discrete physiological parameters. In some embodiments, the processing unit may integrate at least three discrete physiological parameters. In some embodiments, the measured physiological parameters may include such parameters as, but not limited to: breath related parameters (such as, CO₂ measurements in exhaled breath), heart rate related parameters (such as heart rate), blood related parameters (such as, blood pressure), brain activity related parameters (such as brain electrical activity), skin related parameters (such as skin humidity, skin conductivity), body temperature, parameters derived therefrom, or combinations thereof. In some embodiments, at least one of the measured physiological parameters is expired CO₂.

According to some embodiments, there is provided a system for measuring, assessing, determining and/or controlling pain of a subject, the system may include one or more medical monitoring devices configured to measure three or more physiological parameters of a subject, at least one of the measured parameters is expired CO₂; and a processing unit capable of integrating the measured physiological parameters to determine the pain level of the subject based on the measured parameters. According to some embodiments, the additional parameters (in addition to expired CO₂) may be skin humidity, skin conductance, skin capacitance, heart rate, SpO₂, blood pressure, brain activity, data derived therefrom or related thereto, and the like, or any combinations thereof.

In some embodiments, the measured parameters may be further manipulated and/or processed to generate data related to the measurements, prior to or concomitantly while being integrated by the processing unit. For example, in some embodiments, various CO₂ related parameters may be derived from the measurement of expired CO₂, such as, for example but not limited to: CO₂ waveform and parameters related thereto (such as, for example, but not limited to: EtCO2, changes in EtCO₂, a slope of the increase in the CO₂ concentration, a change in a slope of the increase in the CO₂ concentration, time to rise to a predetermined percentage of a maximum value of CO2 concentration, a change in time to rise to a predetermined percentage of a maximum value of CO₂ concentration, an angle of rise to a predetermined percentage of a maximum value of CO₂ concentration, a change in an angle of rise to a predetermined percentage of a maximum value of CO₂ concentration, breath to breath correlation, a change in breath to breath correlation, a CO₂ duty cycle, a change in CO₂ duty cycle, minute ventilation, a change in minute ventilation or any combination thereof), respiration rate, breath cycle, CO₂ concentration in expired air, and the like, or any combination thereof. For example, in some embodiments, various parameters (data) derived from pulse oximetry measurements may be obtained, such as, heart rate (pulse rate), amplitude of cardiac pulses (as determined based on the photoplethysmogram signal); modulation of the amplitude of cardiac pulses, percent modulation (PMod) of the signal, changes in the PMod, and the like, or combinations thereof.

According to some embodiments, the system may further include a signaling unit. The signaling unit may be configured to issue an alert if the pain level of the subject increases over a predetermined threshold or over a period of time (that may be predetermined). The signaling unit may be associated (directly or indirectly) with and controlled by the processing unit. In some embodiments, the signaling unit may further control a pain medication (pain management) subunit, that may affect the amount (concentration and/or administration period) of sedative substances administered to the patient. In some embodiments, the signaling unit automatically controls the administration of sedative agents, based on the determined pain level and/or changes thereto.

According to some embodiments, the measured physiological parameters may be determined or calculated over a period of time, in order to determine change in pain levels over time. The period of time may be predetermined.

According to some embodiments, the pain level may be determined by various calculations and algorithms and may be assigned a value. In some embodiments, the pain level value may be calculated by various means, such as, for example, by use of mathematical equations, algorithms, formulas, and the like, that may take into consideration the values or derivatives of the values of the physiological parameters measured by the one or more monitoring devices.

According to some embodiments the system may include a user interface that may allow the user to select the data to be displayed and to control various operating parameters. Moreover, different displays may be included to accommodate different needs of the different users (such as a nurse, a physician, an anesthesiologist, and the like).

According to some embodiments, the system may further include one or more displays that may be used to present the data collected and determined by the processing logic, the change over time of the data, the determined pain level, the change over time of the pain level, the amount of sedative substances used, and the like.

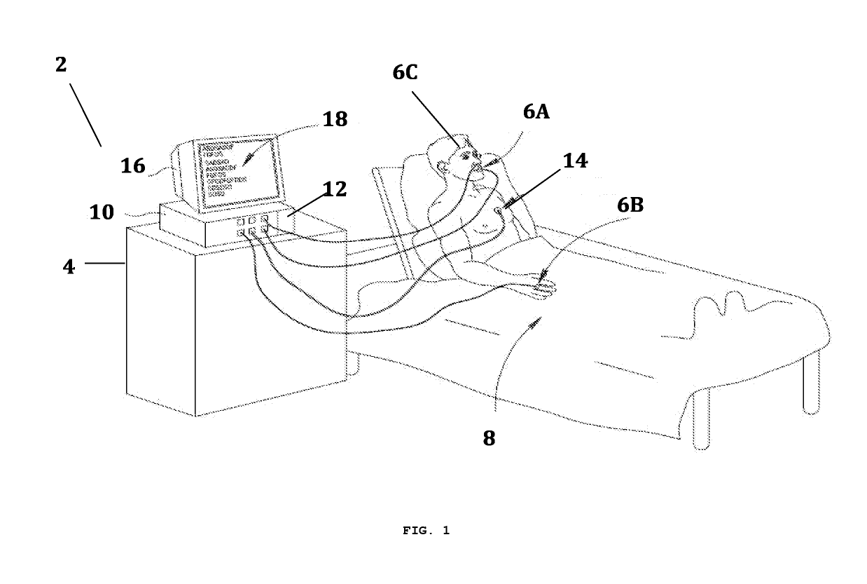

Reference is now made to FIG. 1, which is a schematic illustration of a system, according to some embodiments. As shown in FIG. 1, system (2) may include one or more medical monitoring devices (shown as one integrated device (4)). The medical monitoring devices may include, for example, but not limited to: a capnograph, a pulse oximeter, spirometer, heart rate sensors, blood pressure sensors, ECG, EEG, Ultrasound, thermometer, skin humidity or conductivity measuring device, and the like. The monitoring devices include various sensors, such as, sensors 6A-C that may be configured to obtain/sense/measure various health related parameters of subject (8). The exemplary sensors shown in FIG. 1 are CO₂ sensor (6A), pulse oximetry sensor (6B), and skin humidity sensor (6C). The one or more sensors may be connected directly or indirectly to the patient. The parameters thus measured/obtained/calculated may include, for example, such parameters as, but not limited to: EtCO₂, CO₂ levels, CO₂ waveform pattern, SpO₂, heart rate, blood pressure, blood flow, blood gases, temperature, skin humidity or skin conductance or skin capacitance, and the like. System 2 may further include a processing logic, such as, for example, processing logic 10, that may be used to receive information from the sensors and to process the information to determine pain level of the patient, based on the measured parameters or data derived therefrom. The processing logic may include any type of firmware, hardware and/or software. The connection between the processing logic and the sensor(s) may include any type of communication route, such as, for example, use of wires, cables, wireless, and the like. The system may further include a signaling unit (12) configured to issue an alert if the pain level of the subject increases. The alert issued by the signaling unit may be any type of alert, such as, an audible alert, a visual alert, a tactile alert or combinations thereof. The signaling unit may further be configured to control levels (amount/concentration) and/or administration period of sedative agents administered to the subject. The sedative agents may be administered to the subject by any method known in the art. For example, the sedative agents may be administered by intravenous administration (14). The system may further include one or more displays (such as, for example, display 16 in FIG. 1), that may be used to present the data collected and determined by the processing logic, the change over time of the data, the determined pain level, the change over time of the pain level, the amount of sedative substances used, and the like. In addition, the system may further include a user interface, such as, user interface 18 that may allow the health care provider to input patient related data and/or control any of the operating parameters of the system. The system may further include data storing subunits, capable of string

According to some embodiments, there is provided a method for determining, assessing and optionally managing pain levels of a subject. In some embodiments, the method may include obtaining measurements of one or more physiological parameters of the subject, and determining, based on the measured parameters (or date obtained therefrom), the pain level of the subject or changes to the pain level. In some embodiments, the method may further include issuing an alert if the pain levels increase over a time period or over a predetermined threshold. In some embodiments, the method may further include managing pain level by controlling the level of sedatives administered to the subject.

According to some embodiments, there is provided a method for measuring determining, assessing and optionally managing pain levels of a subject, the method may include obtaining measurements of two or more physiological parameters of the subject, and determining, based on the measured parameters (or date obtained therefrom), the pain level of the subject or changes to the pain level. In some embodiments, the method may include obtaining measurements of three or more physiological parameters of the subject. In some embodiments, the physiological parameters, or data derived therefrom, may include such parameters as, for example, but not limited to: breath related parameters (as determined, for example, based on expired CO₂ (measurements of CO₂ in exhaled breath), heart related parameters (such as heart rate), blood related parameters (such as, blood pressure, oxygen saturation, blood gases), brain activity related parameters (such as brain electrical activity), skin related parameters (such as skin humidity or skin conductivity), body temperature, and the like, or combinations thereof. In some embodiments, at least one of the physiological parameters is expired CO₂.

According to some embodiments, there is provided a method for measuring determining, assessing and optionally managing pain levels of a subject, the method may include obtaining measurements of expired CO₂; and one or more additional physiological parameters of the subject; and determining, based on the measured parameters (or date obtained therefrom), the pain level of the subject or changes to the pain level. According to some embodiments, the additional parameters (in addition to expired CO₂) may be skin humidity, heart rate, SpO₂, blood pressure, brain activity, data derived therefrom, and the like.

In some embodiments, the method may further include manipulation and/or processing of the measured parameters, to generate data related to the measurements, prior to or simultaneously while being integrated for determination of pain level. For example, in some embodiments, various CO₂ related parameters may be derived from the measurement of CO₂ in breath, such as, for example but not limited to: CO₂ waveform and parameters related thereto (such as, for example, but not limited to: EtCO2, changes in EtCO₂, a slope of the increase in the CO₂ concentration, a change in a slope of the increase in the CO₂ concentration, time to rise to a predetermined percentage of a maximum value of CO2 concentration, a change in time to rise to a predetermined percentage of a maximum value of CO₂ concentration, an angle of rise to a predetermined percentage of a maximum value of CO₂ concentration, a change in an angle of rise to a predetermined percentage of a maximum value of CO₂ concentration, breath to breath correlation, a change in breath to breath correlation, a CO₂ duty cycle, a change in CO₂ duty cycle, minute ventilation, a change in minute ventilation or any combination thereof), respiration rate, breath cycle, CO₂ concentration in expired air, and the like, or any combination thereof. Such parameters may be indicative of increase in pain. For example, increase in breath rate, changes in breath depth, changes in CO2 waveform, cessation of breath, may be indicative of increased pain.

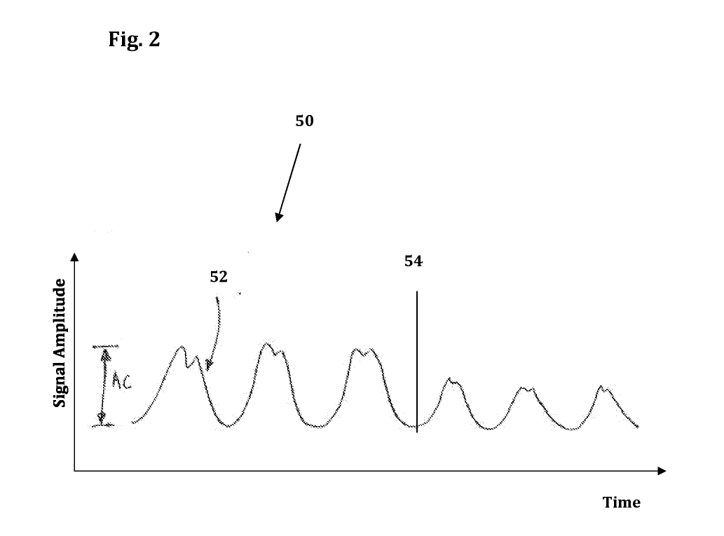

For example, in some embodiments, various parameters (data) derived from pulse oximetry measurements may be obtained, such as, heart rate (pulse rate), amplitude of cardiac pulses (as determined based on the photoplethysmograph signal (PPG)); modulation of the amplitude of cardiac pulses, percent modulation (PMod) of the signal, changes in the PMod, and the like, or combinations thereof. For example, rapid vasoconstriction is a known response to pain. Such vasoconstriction may be identifiable in a photoplethysmograph signal from a pulse oximeter device as a reduction in the amplitude of the pulsatile component. Reference is now made to FIG. 2, which depicts a graph of amplitude of cardiac pulses in a photoplethysmograph signal over time. As shown in FIG. 2, graph (50) shows the amplitude (AC) of cardiac pulses (52) over a period of time. At time point (54), a reduction in the amplitude of the cardiac pulses is observed, which is indicative of the presence of pain. As the amplitude of the cardiac pulses is further reducing over time (beyond time point 54), it is indicative that pain levels are increasing over time.

According to some embodiments, the amplitude of the photoplethysmograph signal may be used as an indication to the pain level or changes thereto. According to some embodiments, the amplitude of the cardiac pulses may further be manipulated and may be divided by the baseline of the signal (i.e. DC level), to produce a normalized measure of pain. In some embodiments, the normalized measure of pain is the percent modulation (PMod) of the signal.

According to some embodiments, the measured physiological parameters may be determined or calculated over a period of time, in order to determine change in pain levels over time. The period of time may be predetermined.

According to some embodiments, the pain level may be determined by various calculations and algorithms and may be assigned a value. In some embodiments, the pain level value may be calculated by various means, such as, for example, by use of mathematical equations, algorithms, formulas, and the like, that may take into consideration the values or derivatives of the values of the physiological parameters measured by the one or more monitoring devices.

According to some embodiments, learning and optimization of determination of pain level may be carried out using various methods, such as, for example, but not limited to: neural networks, a support vector machine (SVM), genetic algorithms, simulated annealing and expectation-maximization (EM), learning systems based on historic data, and the like.

According to some embodiments, there is provided a method used system for measuring, assessing, determining and/or controlling pain of a subject, the system may include one or more medical monitoring devices configured to measure three or more physiological parameters of a subject, at least one of the measured parameters is expired CO₂; and a processing unit capable of integrating the measured physiological parameters to determine the pain level of the subject based on the measured parameters. According to some embodiments, the additional parameters (in addition to expired CO₂) may be skin humidity, heart rate, SpO₂, blood pressure, brain activity, data derived therefrom or related thereto, and the like, or any combinations thereof.

According to some embodiments, there is provided a method for measuring, assessing, determining and/or controlling pain of a subject, the method may include one or more of the steps of: obtaining two or more physiological parameters of a subject, at least one of the measured parameters is expired CO₂; and processing the measured physiological parameters or data derived therefrom to determine the pain level of the subject, based on the measured parameters. According to some embodiments, the additional parameters (in addition to expired CO₂) may be skin humidity, skin conductance, skin capacitance, heart rate, SpO₂, blood pressure, brain activity, data derived therefrom or related thereto, and the like, or any combinations thereof. In some embodiments, the method may further include a step of issuing an alert if the pain level is determined to increase over a predetermined value or over a time period. In some embodiments, the method may further include a step of controlling a pain management subunit configured to administer sedative substances to the patient. In some embodiments, the physiological parameters of the subject are obtained from one or more monitoring medical devices. In some embodiments, the processing of the measured physiological parameters or data derived therefrom and determining of pain levels is performed at a processor. In some embodiments, the issuance of an alert is performed by a signaling unit, configured to issue an audible alert, visual alert or tactile alert.

According to some embodiments, there is provided a method for measuring, assessing, determining and/or controlling pain of a subject, the method may include one or more of the steps of: obtaining three or more physiological parameters of a subject, at least one of the measured parameters is expired CO₂; and processing the measured physiological parameters or data derived therefrom to determine the pain level of the subject, based on the measured parameters. According to some embodiments, the additional parameters (in addition to expired CO₂) may be skin humidity, skin conductance, skin capacitance, heart rate, SpO₂, blood pressure, brain activity, data derived therefrom or related thereto, and the like, or any combinations thereof. In some embodiments, the method may further include a step of issuing an alert if the pain level is determined to increase over a predetermined value or over a time period. In some embodiments, the method may further include a step of controlling a pain management subunit configured to administered sedative substances to the patient. In some embodiments, the physiological parameters of the subject are obtained from one or more monitoring medical devices. In some embodiments, the processing of the measured physiological parameters or data derived therefrom and determining of pain levels is performed at a processor. In some embodiments, the issuance of an alert is performed by a signaling unit, configured to issue an audible alert, visual alert or tactile alert.

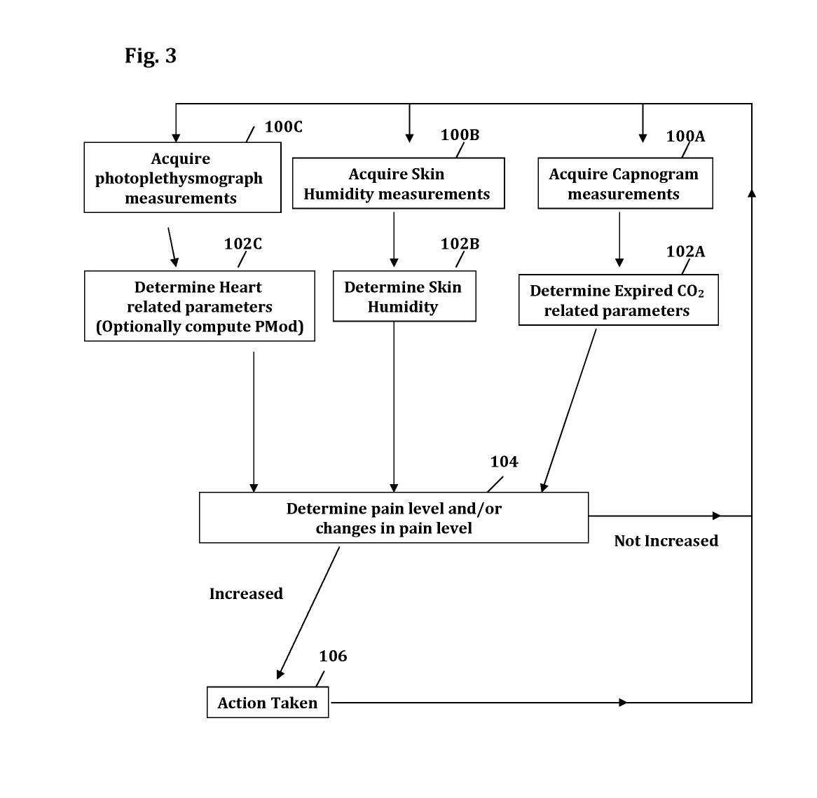

Reference is now made to FIG. 3, which is a schematic block diagram of steps in a method for determining/measuring/managing pain levels of a subject, according to some embodiments. As shown in FIG. 3, measurements of at least three physiological parameters of a subject are obtained from the appropriate corresponding monitoring devices. For example, at step 100A, capnograph measurements are obtained, at step 100B, Skin humidity (skin conductance or skin capacitance) measurements are obtained, and at step 100C, photoplethysmograph (pulse oximetry) measurements are obtained. Each of steps 100A-C may be performed simultaneously, sequentially or independently of each other. Next, the obtained measurements may be further processed or analyzed to determine various related parameters. For example, at step 102A, expired CO₂ related parameters are determined, based on the capnograph measurements. Exemplary CO₂ related parameters include such parameters as, but not limited to: CO₂ waveform and parameters related thereto (such as, for example, but not limited to: EtCO₂, changes in EtCO₂, a slope of the increase in the CO₂ concentration, a change in a slope of the increase in the CO₂ concentration, time to rise to a predetermined percentage of a maximum value of CO₂ concentration, a change in time to rise to a predetermined percentage of a maximum value of CO₂ concentration, an angle of rise to a predetermined percentage of a maximum value of CO₂ concentration, a change in an angle of rise to a predetermined percentage of a maximum value of CO₂ concentration, breath to breath correlation, a change in breath to breath correlation, a CO₂ duty cycle, a change in CO₂ duty cycle, minute ventilation, a change in minute ventilation or any combination thereof), respiration rate, breath cycle, CO₂ concentration in expired air, and the like, or any combination thereof. For example, at step 102B, skin humidity is determined based on skin conductance, skin capacitance, or direct skin humidity measurements (as performed in step 100B). For example, at step 102C, various heart related parameters are determined, based on the pulse oximetry measurements. Exemplary heart related parameters may be such parameters as, but not limited to: heart rate, heart rate (pulse rate), amplitude of cardiac pulses; modulation of the amplitude of cardiac pulses, percent modulation (PMod) of the signal, and the like, and combinations thereof. Each of steps 102A-C may be performed simultaneously, sequentially or independently of each other. Next, at step 104, the various parameters that have been determined in steps 102A-C are integrated (for example, by a processing unit) to determine pain level and/or changes thereto. In some embodiments, the change in the pain level may be compared to a previous value of pain level (as determined over one or more previous cycles). In some embodiments, the change in the pain level is determined by comparing to a reference predetermined value of pain level. Further in step 104, if it is determined that pain level has not increased, a new cycle of measurements and determination of pain levels based thereupon proceeds. If it is determined at step 104 that pain level has increased (over a period of time or over a reference value), action is taken in step 106. The action taken in step 106 may include various actions. For example, at step 106, an alert may be issued. The alert may be any type of alert, such as, for example, an audible alert (such as an alarm sound), a visual alert (such as a flashing light), a tactile alert (such as vibration). The alert may indicate to the health care provider that increasing pain has been detected and that action may be taken. In addition to, or alternatively to the alert issued, an additional action may be to control sedative substances administered to the subject, for example, by automatically controlling a pain management subunit configured to control/provide sedatives to the subject. In some embodiments, the control of the sedative administered to the subject may be on the amount (concentration) of the sedative administered, length of time period over which the sedatives are administered, or both.

According to some embodiments, the systems and methods disclosed herein provide for a non-invasive assessment of pain of a subject.

According to some embodiments, the systems and methods disclosed herein provides for an automatic management of pain of a subject, by controlling a pain medication subunit that may determine the amount of sedative substances administered to the patient.

It is understood by the skilled in the art that the processor of the system is configured to implement the method as essentially described herein.

In the description and claims of the application, each of the words “comprise” “include” and “have”, and forms thereof, are not necessarily limited to members in a list with which the words may be associated.

While a number of exemplary aspects and embodiments have been discussed above, those of skill in the art will recognize certain modifications, permutations, additions and sub-combinations thereof. It is therefore intended that the following appended claims and claims hereafter introduced be interpreted to include all such modifications, permutations, additions and sub-combinations as are within their true spirit and scope.

### EXAMPLES

### Example 1

### Assessment of Pain Level Based on Measured Physiological Parameters

Change in Pain levels of a test subject are determined based on measurements of the following physiological parameter: expired CO₂ (as determined by capnograph measurements), heart rate (as determined by pulse oximetry measurements) and skin humidity (as determined by measurement of skin capacitance).

The subject is continuously being monitored by a capnograph, a pulse oximeter, and skin capacitance sensor. When pain levels of the subject increases, a pattern of rapid breathing is identified based on changes to the CO₂ waveform and frequency of the breath cycles, as identified based on the capnography signal. Additionally, increase in heart rate and reduction in PMod (calculated by dividing the amplitude of the cardiac pulses (obtained based on the photoplethysmograph signal) by the baseline of the signal), are observed. Further, increase in skin humidity is detected (based on increase in skin capacitance). The integrated, combined analysis of the identified changes in the measured parameters and the data derived therefrom, provide a reliable, robust and accurate indication of an increase in the pain level of the subject, and allow the accurate monitoring the pain levels.

The examples described above are non-limiting examples and are not intended to limit the scope of the disclosure. The described examples may comprise different features, not all of which are required in all embodiments of the disclosure.

What is claimed is:

1. A system for determining pain level of a subject, the system comprising:
one or more medical monitoring devices configured to measure three or more physiological parameters of the subject, at least one of said parameters is expired CO₂;
a processing unit configured to integrate the one or more physiological parameters of the subject, to determine the pain level of the subject based on the measured parameters; and
a signaling unit configured to issue an alert if the pain level of the subject increases.

2. The system of claim 1, wherein the medical monitoring device comprises: capnograph, pulse oximeter, skin humidity measurement device, skin conductance measurement device, skin capacitance measurement device Electrocardiogram (ECG), Brain activity monitoring device, or combinations thereof.

3. The system of claim 1, wherein the physiological parameters of the subject comprises: skin humidity, skin conductance, skin capacitance, heart related parameters, blood related parameters, brain electrical activity, parameters derived therefrom, and combinations thereof.

4. The system of claim 1, wherein the expired CO₂ related parameters comprises: CO₂ concentration in breath, EtCO₂, CO₂ waveform, Respiration rate, and combinations thereof.

5. The system of claim 3, wherein the heart related parameters comprises heart rate, amplitude of cardiac pulses, Percent Modulation (PMod) of the cardiac pulses, or combinations thereof.

6. The system of claim 3, wherein the blood related parameters comprises blood pressure, SpO₂ or both.

7. The system of claim 1, wherein increase of the pain levels is determined over a period of time and/or if the pain levels are determined to be higher than a predetermined threshold.

8. The system of claim 1, wherein the alert issued by the signaling unit comprises an audible alert, a visual alert, a tactile alert or combinations thereof.

9. The system of claim 1, wherein the signaling unit is further configured to control levels of sedative substances administered to the subject.

10. The system of claim 1, further comprising a display unit configured to display one or more of: the measured parameters, the determined pain level, a change of one or more of measured parameters over time, a change of the determined pain levels over time, parameters derived from the measured parameters, parameters derived therefrom or combinations thereof.

11. A method for determining pain levels of a subject, the method comprising:
a) obtaining a measurement of three or more health related parameters of the patient, at least one of said parameters in expired CO₂;
b) determining the pain level of the patient based on the measured parameters of the subject; and
c) issuing an alert if the pain levels increases over a time period and/or over a predetermined threshold.

12. The method of claim 11, wherein the physiological parameters of the subject comprises: skin humidity, skin conductance, skin capacitance, heart related parameters, blood related parameters, brain electrical activity, parameters derived therefrom, and combinations thereof

13. The method of claim 11, wherein the expired CO₂ related parameters comprises: CO₂ concentration in breath, EtCO₂, CO₂ waveform, Respiration rate, and combinations thereof.

14. The method of claim 12, wherein the heart related parameters comprises heart rate, amplitude of cardiac pulses, Percent Modulation (PMod) of the cardiac pulses, or combinations thereof.

15. The method of claim 12, wherein the blood related parameters comprises blood pressure, SpO₂ or both.

16. The method of claim 11, wherein the alert comprises an audible alert, a visual alert, a tactile alert or combinations thereof.

17. The method of claim 11 further comprising a step of controlling the amount of sedative agents administered to the subject, based on the determined pain levels.

18. The method of claim 11, further comprising displaying the health related parameters and/or the measured pain levels.

19. The method of claim 11, wherein the measurements of the physiological parameters are obtained from one or more medical monitoring devices.
